(19) United States
(12) Patent Application Publication
(10) Pub. No.: US 2026/0183497 A1
(43) Pub. Date: Jul. 2, 2026

(54) SMART INHALER DEVICE FOR MEDICATION ADHERENCE

(71) Applicant: RxCap, Inc., Lynnfield, MA (US)

(72) Inventors: Sreeram Ramakrishnan, Lynnfield, MA (US); Michael Furlotti, Ross, CA (US); Ram Subramanian, Fremont, CA (US)

(21) Appl. No.: 19/438,358

(22) Filed: Dec. 31, 2025

Related U.S. Application Data
(60) Provisional application No. 63/740,460, filed on Dec. 31, 2024.

(51) Int. Cl. A61M 15/00

(52) U.S. Cl. CPC A61M 15/009; A61M 15/0025; A61M 15/008; A61M 2202/0007; A61M 2202/04; A61M 2205/12; A61M 2205/3327; A61M 2205/3553; A61M 2205/3576; A61M 2205/50; A61M 2205/581; A61M 2205/582; A61M 2205/583; A61M 2210/0625

(57) ABSTRACT

An inhaler tracking device for use with an inhaler or cannister configured to release a substance upon actuation includes a housing including a chamber for receiving the inhaler or cannister, the housing configured to hold the inhaler or cannister at a predetermined orientation, plural sensors sensing plural conditions or events including those of the inhaler or cannister, at least one indicator providing feedback to a user, and a controller configured to receive data from the plural sensors, analyze dispensing from the inhaler or cannister based on the received data, and output feedback signals to the at least one indicator. The analyzing includes determining duration and/or intensity of shaking of the device prior to dispensing, orientation of the device during dispensing, and number of actuations of the inhaler or cannister.

### CROSS-REFERENCE TO RELATED APPLICATIONS

This application claims the benefit under 35 U.S.C. § 119(e) of U.S. Provisional Patent Application No. 63/740,460, filed Dec. 31, 2025, the entirety of which is incorporated herein by reference.

### FIELD OF THE INVENTION

The invention relates to a smart device for medication inhalers and a system and method of its use. The device, system, and method of the present invention can use LTE and/or Bluetooth® communication, for example, and are designed to be used with and attached to a known medication canister that can discharge a medicinal inhalant to a patient.

### BACKGROUND OF THE INVENTION

Medication non-compliance is a major problem in health care. Medications in the form of a spray or mist, are typically provided to a user in a disposable inhaler. An inhaler is a small, handheld device that delivers medication directly to the patient's lungs. Types of inhalers include dry powder, metered-dose, and soft mist. Inhalers commonly deliver medication that helps open airways or reduce inflammation in the lungs. Typically, the medication is delivered by metered dose inhalers in unit doses in the form of an aerosol. Each actuation of the inhaler delivers one unit dose. The unit dose is expelled by the inhaler and is taken into the body of the patient upon inhalation, via the nose or mouth. The pharmaceutical formulation is delivered to or via the respiratory track, notably to the lungs, of the patient on inhalation.

When physicians prescribe inhalant medications, they typically advise the patients of a proper medication administration, such as to take the medication at appropriate times in appropriate quantities and in the appropriate manner, to continue taking the medication for the full prescribed regimen, even if the patient feels better, etc. Unfortunately, many patients exhibit poor compliance in properly following the regimens set out by their physicians, including regimens that require inhalers.

The effect of an inhaled medicant is dependent on the ability of the patient to inhale the right amount of the medication in the proper manner. For example, some patients with asthma do not use their inhaler properly and may not receive the correct amount of medication upon inhalation. This can be due to a lack of hand-breath coordination (press and breathe), an incorrect speed and/or depth of inhalation, an incorrect actuation of the canister, and a missing breath-hold after inhalation of the dose of medicament. There is a need to implement guiding functions in the metered dose inhaler in order to help patients to properly use their inhaler.

A variety of products and techniques for reminding patients to take their medications, as prescribed, are known. Some compliance intervention systems offered by health care providers are designed to remind the patient to take the medication and alert a remote caregiver if the patient does not comply with taking the medication as prescribed. Some of these compliance intervention systems include sensors/reminders in the home, a network connection, and outbound messaging to a caregiver or even back to the patient.

Various attempts have been made to try to increase and improve the compliance of patients in the taking of their medications. Most of these systems are reminder systems

### SUMMARY OF THE INVENTION

In one aspect, the invention relates to an inhaler tracking device for use with an inhaler or cannister containing a substance to be inhaled, actuation of the inhaler or cannister causing the cannister to release the substance from the cannister through a passageway to a mouthpiece, the inhaler tracking device comprising a housing including a chamber for receiving the inhaler or cannister, the housing configured to hold the inhaler or cannister at a predetermined orientation; plural sensors sensing plural conditions or events including those of the inhaler or cannister; at least one indicator providing feedback to a user; and a controller configured to receive data from the plural sensors, analyze dispensing from the inhaler or cannister based on the received data, and output feedback signals to the at least one indicator, wherein analyzing dispensing from the inhaler or cannister includes determining duration and/or intensity of shaking of the device prior to dispensing, orientation of the device during dispensing, and number of actuations of the inhaler or cannister.

In another aspect, the invention relates to an inhaler tracking device for use with an inhaler or cannister containing a substance to be inhaled, actuation of the inhaler or cannister causing the cannister to release the substance from the cannister through a passageway to a mouthpiece, the inhaler tracking device comprising a housing including a chamber for receiving the inhaler or cannister, the housing configured to hold the inhaler or cannister at a predetermined orientation; plural sensors sensing plural conditions or events including those of the inhaler or cannister; at least one communication device; and a controller configured to receive data from the plural sensors, analyze dispensing from the inhaler or cannister based on the received data, and output feedback signals through the communication device, wherein analyzing dispensing from the inhaler or cannister includes determining duration and/or intensity of shaking of the device prior to dispensing, orientation of the device during dispensing, and number of actuations of the inhaler or cannister.

In a further aspect, the invention relates to a method of tracking use of an inhaler or cannister containing a substance to be inhaled, actuation of the inhaler or cannister causing the cannister to release the substance from the cannister through a passageway to a mouthpiece, the method comprising sensing plural conditions including those of the inhaler or cannister; and receiving data from the plural conditions or events, analyzing dispensing from the inhaler or cannister based on the received data, and outputting feedback signals to at least one indicator to provide feedback to a user, wherein analyzing dispensing from the inhaler or cannister includes determining duration and/or intensity of shaking of the inhaler or cannister prior to dispensing, orientation of the inhaler or cannister during dispensing, and a number of actuations of the inhaler or cannister.

These and other aspects of the invention will become apparent from the following disclosure.

### BRIEF DESCRIPTION OF THE DRAWINGS

FIG. 1 is a side view of an embodiment of the inhaler tracking device of the present invention with an inhaler received therein.

FIG. 2 is a side view of another embodiment of the inhaler tracking device of the present invention with an inhaler received therein.

FIG. 3 is a side view of yet another embodiment of the inhaler tracking device of the present invention with an inhaler received therein.

FIG. 4 is a perspective view of the inhaler tracking device of the present invention.

FIG. 5 is another perspective view of the inhaler tracking device of the present invention.

FIG. 6 is a schematic view of the control system of the inhaler tracking device of the present invention.

### DETAILED DESCRIPTION OF THE PREFERRED EMBODIMENTS

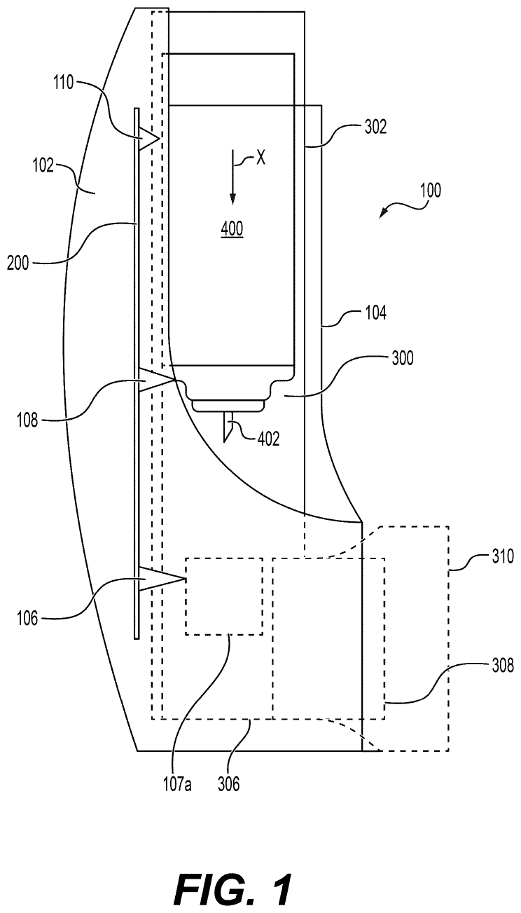

Various embodiments of an inhaler tracking device 100 according to the present invention are shown in FIGS. 1-5. The device 100 is designed to be used with and attached to a known medication inhaler 300 that can discharge a medicinal inhalant for inhalation by a patient. Alternatively, the device 100 can include several features of an inhaler and need only receive a canister or cartridge 400. Both scenarios will be discussed below.

A known metered dose inhaler 300 comprises an actuator housing 302 adapted to receive an aerosol container 400 with an activation valve 402 at a valve end of the container. The container 400 is configured to move in a longitudinal direction X from a rest position to an activation position in which the valve 6 is depressed against an unshown trigger portion of the actuator housing 302 such that an aerosol dose is released. An inhalation flow through the actuator housing 302 is directed to a mouthpiece 308 of the metered dose inhaler located at a front side of the actuator housing 302. A mouthpiece cap 310 can cover the mouthpiece when not in use and is removed when the inhaler is to be used by a patient.

Inhaler tracking device 100 is configured to receive inhaler 300 and, keep track of its use and provide feedback to a user and/or a remote receiving station. Inhaler tracking device 100 includes, in one embodiment, a housing 102 configured in size and shape to receive and hold inhaler 300 and a clamp 104 used to press and secure the inhaler 300 to the housing. The housing 102 and clamp 104 can use any known connection means. When inhaler 300 is secured to housing 102, the inhaler can be used in its intended manner.

Inhaler tracking device 100 is provided with a control system and plural sensors as well as additional components to achieve desired operation and performance of its smart features. The control system includes a controller 200 in the form of, for example, a printed circuit board (PCB)or printed circuit board assembly (PCBA) as well as appropriate memory, such as a read only memory (ROM) and/or a random access memory (RAM), to control the device, a communications module, such as 5G cellular module or a Bluetooth® module (or other wired or wireless module) for remote communication, a global positioning system (GPS) module, an accelerometer, various sensors such as temperature and humidity sensors, a pressure sensor, and contact sensors, a battery, such as a rechargeable battery, lighting unit(s) such as light emitting diodes (LEDs), a piezoelectric element/device or speaker for audible alerts, one or more buttons or switches, and a display such as an LCD display.

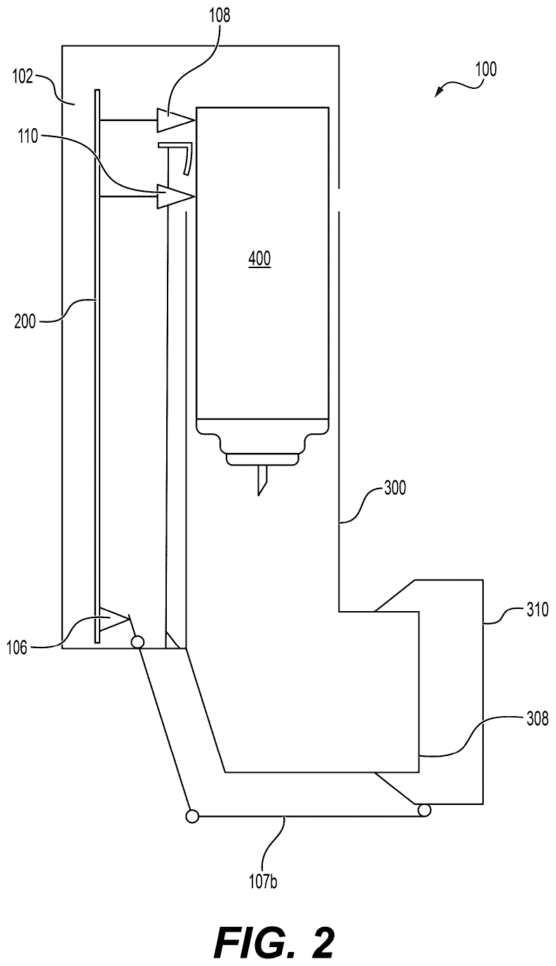

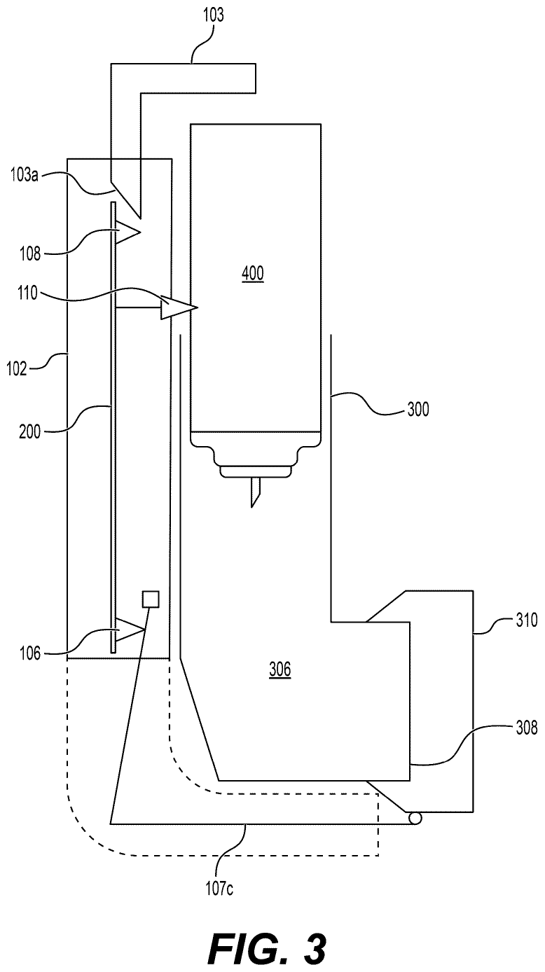

The foregoing sensors will now be described in more detail. A mouthpiece cap sensor 106 is configured to detect whether the mouthpiece cap 310 is in a capping position or an uncapped position. In the embodiment of FIG. 1, a cap actuator 107 is associated with movement of the cap 310. When in the position of FIG. 1, the actuator 107a engages a switch of sensor 106 and the controller determines that the cap 310 is in the capping position. Conversely, when the cap is removed, the actuator 107a and switch 106 are disengaged, the controller determines that the cap is in the uncapped position. In the embodiments of FIGS. 2 and 3, a linkage 107b or 107c can be associated with movement of the cap 310 to perform the cap position detecting function. Of course, any other know type of sensor can be used, contacting or contactless, including light sensors.

A dispensing or puff sensor 108 is disposed near inhaler 300 to detect when the inhaler has been depressed to release medicant mist. Sensor 108 can be in the form of a switch that engages an edge of cannister 400, for example, as shown in FIG. 1. Alternatively, sensor 108 can engage a sidewall of cannister 400 near its distal end and disengage when the switch no longer contacts the sidewall because of the dispensing movement, as shown in the embodiment of FIG. 2, or engage a cam 103a connected to a depressing handle 103 and activate when the handle is depressed to release medicant, as shown in the embodiment of FIG. 3. Of course, any other know type of sensor can be used, contacting or contactless, including light sensors or even sensors that can determine the length of the depression stroke.

An inhaler mounting sensor 110 is disposed near inhaler 300 and is configured to detect when the inhaler 300 has been mounted or inserted in the housing 102 of the inhaler tracking device 100. Sensor 110 can also be in the form of a switch that engages an edge of inhaler 300, for example, as shown in FIG. 1, or that engages a wall of cannister 400, as shown in FIGS. 2 and 3. When the switch is activated, the controller determines that the inhaler 300 or cannister 400 is properly mounted. Of course, any other know type of sensor can be used, contacting or contactless, including light sensors.

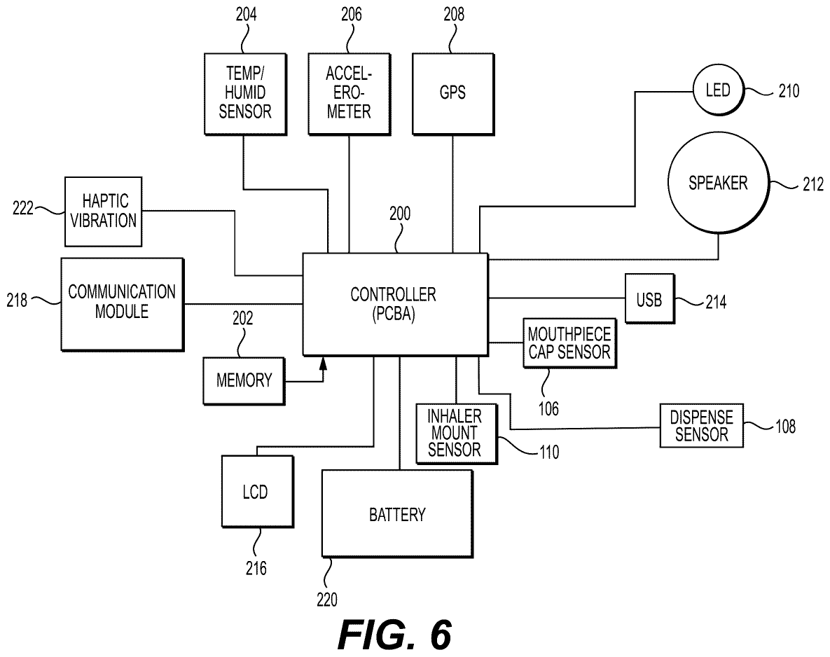

FIG. 6 is a schematic diagram of the foregoing electrical/electronic components of the system. As noted above, controller 200 of the inhaler tracking device 100 can be in the form of a PCB, which receives signals from and transmits signals to the several electrical components of the smart inhaler tracking device 100. Controller 200 is provided with any suitable memory 202 that it can use as a workspace and to store and retrieve data and programs. The inhaler tracking device 100 can be provided with a temperature and/or humidity sensor 204, an accelerometer 206, and a global positioning system (GPS) unit 208 in communication with controller 200. These sensors can be of any configuration known to those in the art. The temperature and/or humidity sensor 204 can sense ambient temperature and/or humidity conditions of the smart inhaler tracking device 100 and can convert those conditions into an electrical signal to supply to controller 200. Any other sensors or input devices can be included. For example, a sensor that can measure the air quality index or receive such information can be used to alert the user for potential use of the inhaler. This feature can be particularly useful for rescue inhalers for asthmatic patients.

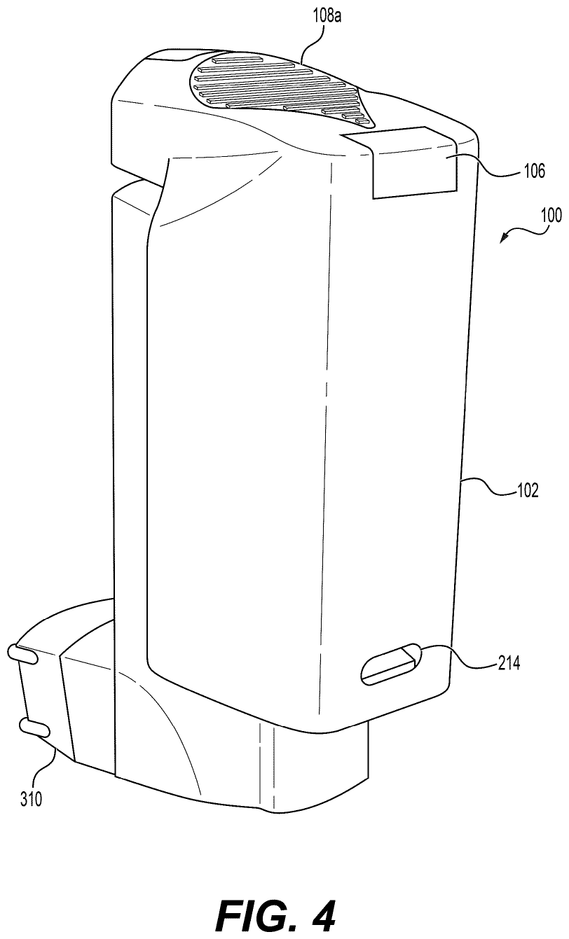

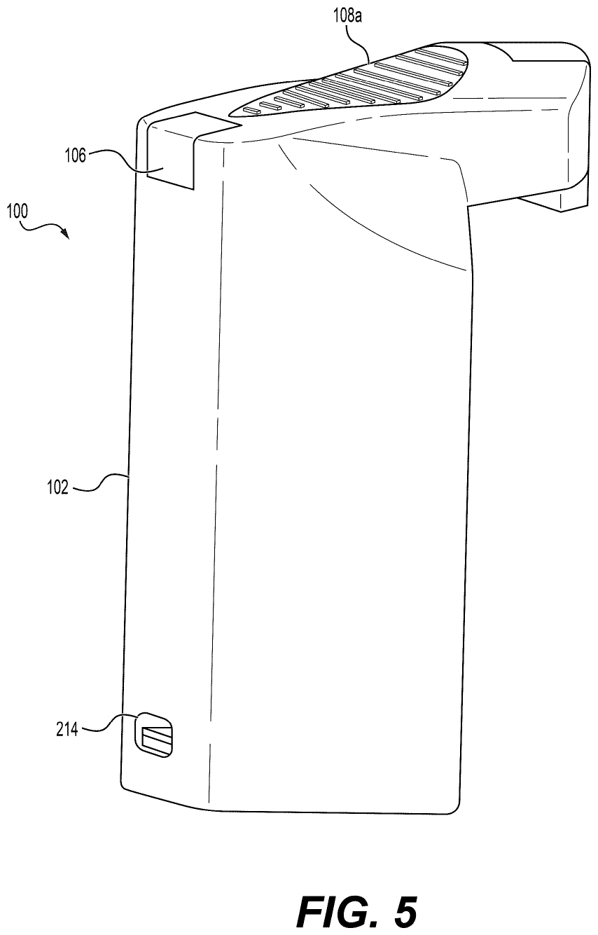

The inhaler tracking device 100 may also have one or more LEDs 210 placed thereon. The LEDs may be illuminated in order to alert a user when it is time to inhale his or her medication, as will be discussed more fully below. As would be understood by one having ordinary skill in the art, multiple color LEDs may be used. Further, the location of the LEDs is not limited to any particular face of inhaler tracking device 100. The LEDs may be placed at any location in order to alert a user. An example is shown in FIGS. 4 and 5, where the LEDs 210 are disposed on the top of inhaler tracking device 100, above USB port 214 discussed below.

Smart inhaler tracking device 100 can also be provided with an LCD display 216 in addition to, or in place of, the LEDs. LCD display 216 can be positioned on the top of the inhaler tracking device 100, for example, adjacent to the LEDs. The LCD display can perform many functions, such as alerting the user as to when it is time to take his or her medication, indicating the status of the smart inhaler tracking device 100, outputting an error message, and providing dosage instructions.

GPS unit 208 can be used to track the location of the smart inhaler tracking device 100. This data can be used to track the habits of the user.

The smart inhaler tracking device 100 is also provided with a communications module 218 and/or a USB port 214 connected to controller 200. This allows communication with the smart inhaler tracking device 100 remotely or directly. In this manner, any information stored in memory 202 can be downloaded so as to track dispensing times and compliance. These connections can also be used to program the controller when needed, such as when upgrading its software. In addition, USB port 214 can be used to charge rechargeable battery 220 in any known manner.

An alert device will be provided in the smart inhaler tracking device 100 in order to alert a user of the time to take the medication or of errors in the system. The alert device may be an audio alarm, a visual alarm, a vibration alarm, or any combination thereof. The visual alarm may be the light emitting devices (LEDs) 210 or LCD display 216. For example, one of the LEDs glows green when the user is to inhale the medicant and another glows red when it is not yet time for the user to inhale the medicant. The audio alarm will emit an audible signal through speaker 212 when it is time for a user to inhale the medication and a haptic vibrating alarm 222 will vibrate the smart inhaler device 100 when it is time for inhalation.

The visual alarm may be a flashing light or may be a steady light. Further, the audio alarm may emit sound in a pattern, may emit a steady sound or may be an automated voice. Further, the smart inhaler tracking device 100 is not limited to a single type of alert device. The smart inhaler tracking device 100 may contain all three types of alarms, any combination of the three types of alarms, or other alerting devices not discussed herein.

The alarms in the smart inhaler tracking device 100 are not only for alerting a user when to take medication, but can also alert the user if there is a system malfunction. For example, if the battery is getting too low, the smart inhaler tracking device 100 could emit an audio alarm with a sound that differs from the audio alarm sound used to indicate it is time to take medication. Also, the smart inhaler tracking device 100 could emit a different color LED 210 if there is a system malfunction.

The LEDs 210 may also be used to alert the user to what type of medication is in the container 400 in the inhaler 300. As an example, if a user is taking a variety of inhalants, a smart inhaler tracking device 100 for COPD medication could glow red, and a smart inhaler tracking device 100 for asthma medication could glow blue.

The smart inhaler tracking device 100 utilizes communications module 218 to send and receive communications regarding user, prescription, and compliance information. The transceiver may be Zigbee and/or Bluetooth technology, a cell modem, a RFID transmitter, or any other known device for sending and receiving information. Preferably, the smart inhaler tracking device 100 contains more than one transceiver for redundancy. For example, the smart inhaler tracking device 100 preferably contains a cell modem and Bluetooth and/or Zigbee technology.

The communications module will allow the controller 200 to send messages, via SMS text messages or any other suitable protocol such as TCP/IP, to a central server so as to report compliance data of a user, any malfunctions, any tampering, or any misuse of the pills that is sensed by the smart inhaler tracking device 100. The Bluetooth or Zigbee technology allows for the device to be able to quickly interact with the pharmacy computing system. The pharmacy computer will detect the smart inhaler tracking device 100 and its unique ID and will download any necessary data to the device.

Information from the cell modem may also be used by an external server to send messages to any outside source, for example, a user's family or friends, a caretaker, doctor, other healthcare provider, a researcher, pharmaceutical company, a pharmacy for refills, etc., as needed or desired.

The control system can also provide training or feedback for proper usage. When the patient is taking medication, the device and system can determine and register:
Medication canister shake using the accelerometer 206. Using accelerometer data, the device and system can detect the time and nature of the shake prior to dispense. The device can warn when the user does not shake or mix the medication as instructed.
Orientation of the inhaler during the administration using the accelerometer 206.
Whether the cap 310 has been removed after the inhaler has been shaken correctly. This can be effected using the mouthpiece cap detection switch 106.
Number of puffs (presses). This can be effected by using the puff switch 108 that is triggered when the medication canister is pushed down past the switch or sensor.
Hold time/Dwell time for medication. This can detect if the user is holding the inhaled medication by detecting accelerometer 206 changes and the mouthpiece switch 106 just after the puff is detected.
Whether the inhaler tracking device 100 is removed from medication canister.
When the cap 310 on the mouthpiece 308 is replaced.

The device also has audio and haptic feedback to help coach patients on:
How long to shake
Number of Puffs
Inhalation, breath hold, and exhalation times based on medication.

The device's audio/visual/haptic feedback along with app or text message will remind patients on their next scheduled dose

The device in combination with weather data, real time air quality monitors and the RxCap data hub will provide reminders, alerts, and coaching to remind patients to keep/take the inhaler with them or to take medication based on environmental conditions.

The device can be used in conjunction with devices such as activity trackers, pulse oximeters, and environmental sensors, as well as with usage patterns (and/or behavior patterns) to provide alerts and warnings that may aid in preempting episodes (e.g., to remind patients to take the inhalers with them).

Warnings and alerts can be sent to caregivers, parents, and/or providers when the device is used by the patient.

The device and system can also analyze device usage and prescription information to provide refill reminders.

Another embodiment includes attaching the entire device 100 to the canister 400. In this embodiment, an inhaler is not used, but rather the inhaler tracking device would include the previously-described features of the inhaler, such as the housing 302, mouthpiece 308, and cap 310.

Although this invention has been described with respect to certain specific exemplary embodiments, many additional modifications and variations will be apparent to those skilled in the art in light of this disclosure. It is, therefore, to be understood that this invention may be practiced otherwise than as specifically described. Thus, the exemplary embodiments of the invention should be considered in all respects to be illustrative and not restrictive, and the scope of the invention to be determined by any claims supportable by this application and the equivalents thereof, rather than by the foregoing description.

What is claimed is:

1. An inhaler tracking device for use with an inhaler or cannister containing a substance to be inhaled, actuation of the inhaler or cannister causing the cannister to release the substance from the cannister through a passageway to a mouthpiece, the inhaler tracking device comprising:
a housing including a chamber for receiving the inhaler or cannister, the housing configured to hold the inhaler or cannister at a predetermined orientation;
plural sensors sensing plural conditions or events including those of the inhaler or cannister;
at least one indicator providing feedback to a user; and
a controller configured to receive data from the plural sensors, analyze dispensing from the inhaler or cannister based on the received data, and output feedback signals to the at least one indicator, wherein
analyzing dispensing from the inhaler or cannister includes determining duration and/or intensity of shaking of the device prior to dispensing, orientation of the device during dispensing, and number of actuations of the inhaler or cannister.

2. The inhaler tracking device according to claim 1, wherein analyzing dispensing from the inhaler or cannister includes determining a dwell time of an inhalation.

3. The inhaler tracking device according to claim 2, wherein determining the dwell time of an inhalation is performed using a dispensing sensor and a mouthpiece cap position sensor.

4. The inhaler tracking device according to claim 1, wherein the plural sensors include an accelerometer, a cap position sensor, and a dispensing sensor.

5. The inhaler tracking device according to claim 1, wherein the at least one indicator includes at least one of visual, audible, and haptic feedback.

6. The inhaler tracking device according to claim 1, wherein the plural sensors and the at least one indicator are controlled by the controller in order to train a user to use the inhaler or cartridge and/or provide usage feedback.

7. The inhaler tracking device according to claim 1, wherein the plural sensors and the at least one indicator are controlled by the controller to track usage of the inhaler or cannister, provide reminders to a user, and/or promote medication adherence.

8. The inhaler tracking device according to claim 1, wherein the passageway and the mouthpiece form part of the inhaler tracking device, and the cannister is replaceably mountable in the housing.

9. An inhaler tracking device for use with an inhaler or cannister containing a substance to be inhaled, actuation of the inhaler or cannister causing the cannister to release the substance from the cannister through a passageway to a mouthpiece, the inhaler tracking device comprising:
a housing including a chamber for receiving the inhaler or cannister, the housing configured to hold the inhaler or cannister at a predetermined orientation;
plural sensors sensing plural conditions or events including those of the inhaler or cannister;
at least one communication device; and
a controller configured to receive data from the plural sensors, analyze dispensing from the inhaler or cannister based on the received data, and output feedback signals through the communication device, wherein
analyzing dispensing from the inhaler or cannister includes determining duration and/or intensity of shaking of the device prior to dispensing, orientation of the device during dispensing, and number of actuations of the inhaler or cannister.

10. The inhaler tracking device according to claim 9, wherein analyzing dispensing from the inhaler or cannister includes determining a dwell time of an inhalation.

11. The inhaler tracking device according to claim 10, wherein determining the dwell time of an inhalation is performed using a dispensing sensor and a mouthpiece cap position sensor.

12. The inhaler tracking device according to claim 9, wherein the plural sensors include an accelerometer, a cap position sensor, and a dispensing sensor.

13. The inhaler tracking device according to claim 9, wherein communication device sends feedback to a central location.

14. The inhaler tracking device according to claim 9, wherein the plural sensors and the communication device are controlled by the controller in order to train a user to use the inhaler or cartridge and/or provide usage feedback.

15. The inhaler tracking device according to claim 9, wherein the plural sensors and the communication device are controlled by the controller to track usage of the inhaler or cannister, provide reminders to a user, and/or promote medication adherence.

16. The inhaler tracking device according to claim 9, wherein the passageway and the mouthpiece form part of the inhaler tracking device, and the cannister is replaceably mountable in the housing.

17. A method of tracking use of an inhaler or cannister containing a substance to be inhaled, actuation of the inhaler or cannister causing the cannister to release the substance from the cannister through a passageway to a mouthpiece, the method comprising:
sensing plural conditions including those of the inhaler or cannister; and
receiving data from the plural conditions or events, analyzing dispensing from the inhaler or cannister based on the received data, and outputting feedback signals to at least one indicator to provide feedback to a user, wherein
analyzing dispensing from the inhaler or cannister includes determining duration and/or intensity of shaking of the inhaler or cannister prior to dispensing, orientation of the inhaler or cannister during dispensing, and a number of actuations of the inhaler or cannister.

18. The method according to claim 17, wherein analyzing dispensing from the inhaler or cannister includes determining a dwell time of an inhalation.

19. The method according to claim 17, wherein analyzing dispensing from the inhaler or cannister is used to train a user to use the inhaler or cartridge and/or provide usage feedback.

20. The method according to claim 17, wherein the analyzing dispensing from the inhaler or cannister is used to track usage of the inhaler or cannister, provide reminders to a user, and/or promote medication adherence.
